(19) United States
(12) Patent Application Publication
(10) Pub. No.: US 2015/0366709 A1
(43) Pub. Date: Dec. 24, 2015

(54) OPHTHALMIC TREATMENT APPARATUS

(71) Applicant: Philip C. ROHOLT, Canton, OH (US)

(72) Inventor: Philip C. ROHOLT, Canton, OH (US)

(21) Appl. No.: 14/307,556

(22) Filed: Jun. 18, 2014

(51) Int. Cl. A61F 9/007

(52) U.S. Cl. CPC A61F 9/00709

(57) ABSTRACT

An ophthalmic treatment apparatus and procedure for removing the epithelium of the cornea of the eye and for foreign body removal from the cornea or conjunctiva using sonic and/or ultrasonic energy, includes a brush for removing the epithelium of the cornea and a motor drive for sonically and ultrasonically vibrating the brush. The motor drive can be included as part of a handle connected to a brush head, and can include a generator for producing either sonic or ultrasonic energy within the handle and a transducer in electrical communication with the generator. The brush has a surface adapted to be applied against and substantially parallel to the cornea. The ophthalmic treatment apparatus can be used in combination with an ophthalmic fixating device which serves as a guide to provide a demarcation line or area for epithelial removal by the brush.

### FIELD OF THE INVENTION

The present invention relates generally to an apparatus and method for removing corneal, epithelial tissue, and more particularly to employing both sonic and ultrasonic energy to assist in the removal of the superficial epithelial layer of the cornea and/or in foreign body removal from the cornea or conjunctiva.

### BACKGROUND OF THE INVENTION

The human cornea comprises a thin, protective epithelial layer, typically about 50 μm thick, on top of the Bowman's membrane or layer, which in turn covets the major corneal stroma. Of particular interest to the present invention, the superficial outer epithelial layer of the cornea is often removed as a precursor to excimer laser photorefractive keratectomy (PRK). A number of techniques have been described to remove the epithelium, including blunt debridement using a surgical blade, alcohol softening (typically followed by blunt debridement), and mechanical debridement with a rotating brush. When the corneal epithelium is removed via any of these techniques it will typically regenerate in about 3-5 days; however, all of these techniques have the potential problems of delayed improvement in visual acuity caused by epithelial defects, significant postoperative pain, and formation of stromal haze.

More specifically, blunt debridement of the corneal epithelium by means of scraping with a surgical blade is an inherently rough, imperfect, and inaccurate procedure, even with the exercise of great skill and care. This technique tends to create nicks and cuts, and ragged edges in the epithelium, and can also damage the underlying Bowman's layer. Blade scraping is also slow and tedious, and the removal area is typically much larger than the area of the eye which will be treated. Such excess, removal of epithelial tissue can increase the time necessary for healing, and can cause significant postoperative pain.

Solvent softening with alcohol or cocaine has also been used as a method for removal of the corneal epithelium. A solvent such as diluted alcohol (e.g. a 15-20% alcohol solution) is capable of softening and dissolving away the epithelium over the central cornea, and then a blade or surgical forceps is typically needed to remove remaining debris material over the central 8-10 mm of the corneal surface. However, alcohol is toxic and can damage the eye tissue even if properly used by someone with skill and experience.

More recently, rotating brushes have been used for removal of the corneal epithelium. For example, U.S. Pat. No. 5,649,943 to Amoils describes the use and structure of a hand-held rotating plastic brush for removal of the epithelial layer from the central cornea, which is fabricated by modifying an electric toothbrush (i.e. the Rotadent® tooth brush). The Amoils rotating brush has been found to remove the corneal epithelial layer more quickly and evenly than blunt debridement. Operating at about 1500 revolutions per minute, and with a typical brush diameter of about 6 mm, the Amoils rotating brush moves almost linearly in a broad arc, which translates to a linear velocity of about 0.5 meters/second or 1.6 feet per second across the corneal surface. With a brush diameter of 8 mm, the Amoils bristle velocity is about 4.0 feet per second. These lower bristle tip velocities have proven safe for treating the corneal surface. Nevertheless, rotating brushes still result in ragged epithelial edges, can cause disruption of LASIK flaps, and it is difficult to precisely control the total epithelial removal area. To ensure that adequate access is provided for the laser, epithelial tissue is typically removed peripherally beyond the laser treatment site, such that regeneration of the epithelial layer and post-operative healing can be delayed.

It is known in the art to use relatively low ultrasonic frequency energy for a wide variety of purposes. Different oscillatory frequencies have been used, in medical devices for various purposes. Electrical dental brushes have been proposed in the low sonic range, as in U.S. Pat. No. 3,535,726 to Sawyer. Others have created a dental brush that operates from 200-500 Hz, as in U.S. Pat. No. 4,787,847 to Martin. Ultrasonic medical devices can operate at a frequency between 5,000 to 1,000,000 Hz, typically at 40,000 Hz, and these frequencies have been used in both dental brushes (see U.S. Pat. No. 3,375,820 to Kuris et al., U.S. Pat. No. 3,335,443 to Parisi et al., and U.S. Pat. No. 3,809,977 to Balamuth et al.) and for ophthalmic phacoemulsifiers (see U.S. Pat. No. 3,589,363 to Banko) to remove cataracts.

The energy of both low-frequency and high frequency ultrasound can be converted to heat, physical forces, and acoustical pressures over an undesirably large area, as opposed to confining the energy delivery to the unwanted material or tissue. In addition to the use of ultrasound to break down tissues physically, ultrasound has been used to kill tissues by the generation of heat. This use of ultrasound to kill or harm cells at a distance from the transducer is commonly referred to as thermotherapy or hyperthermia. See, for example, U.S. Pat. No. 5,460,595 to Hall et. al and U.S. Pat. No. 6,685,657 to Jones.

While known ultrasonic medical devices may be useful for their intended purposes, there currently is no device or method for ultrasonically removing a surface layer of corneal epithelium from the eye, or for an object lodged in the corneal epithelium of the eye. It would thus be beneficial to provide an ultrasound assisted, ophthalmic treatment apparatus utilizing gentle, oscillatory brushes of various sizes that could also operate at different frequencies, depending upon the task at hand. It would also be desirable to provide an improved apparatus and method for removing the epithelial layer from the cornea without damaging the Bowman's layer or the underlying stroma. It would further be desirable if this improved device and method provided highly reliable and repeatable results without delaying surgical time, without removing excess epithelium, and without causing ragged epithelial edges or scarred epithelium.

### SUMMARY OF THE INVENTION

The present invention solves the problems related to removing the superficial corneal epithelial tissue and for foreign body removal fours the cornea or conjunctiva, and provides a brush head that can be both socially and ultrasonically powered. The brush can be used in combination with a fixation device or guide, and allows for gentle disruption and easy removal of corneal epithelial tissue without causing scarring or disruption of the underlying Bowman's or stromal layers, and without removing excess epithelium peripherally beyond the laser treatment site.

One aspect of the invention provides an ophthalmic treatment apparatus for removing the superficial corneal epithelium layer of the eye, the apparatus comprising (a) a brush for removing the superficial corneal epithelium layer, the brush having a surface adapted to be applied against and substantially parallel to the cornea; and (b) a motor drive for sonically or ultrasonically vibrating the brush during operation. Typically the motor drive includes a generator capable of producing both sonic and ultrasonic energy waves (such as with a piezoelectric motor), a transducer in electrical communication with the generator, and a power source for providing power to the generator.

Another aspect of the invention provides an ophthalmic treatment apparatus for removing the superficial corneal epithelium layer of the eye, comprising (a) a brush for removing the superficial corneal epithelium layer and/or for foreign body removal from the cornea or conjunctiva, the brush having a surface adapted to be applied against and substantially parallel to the cornea; (b) a motor chive for sonically or ultrasonically vibrating the brush during operation; and (c) an ophthalmic fixating device for stabilizing the eye and for providing a demarcation line/area for epithelial removal by the brush.

Another aspect of the invention provides a procedure for using both sonic and ultrasonic energy to assist in the removal of the superficial corneal epithelial layer of an eye, comprising the steps of (a) exposing a corneal surface of the eye; (b) using sonic and ultrasonic energy to vibrate a surface of a brush; and (c) applying the surface of the brush to the corneal surface of the eye until the superficial corneal epithelial layer has been removed, while leaving the underlying corneal stroma layer and Bowman's layer intact.

The ophthalmic fixating device typically includes a handle connected to a fixation ring, with the fixation ring including a bottom portion and a top rotatable ring portion and a clamp for reversibly connecting the bottom and top portions. The inside edges of the top rotatable ring portion can oppose the corneal epithelium and define an internal diameter which delineates the demarcation line/area of epithelial removal.

The nature and advantages of the present invention will be more fully appreciated after reviewing the accompanying drawings, detailed description and claims.

### BRIEF DESCRIPTION OF THE DRAWINGS

The accompanying drawings illustrate embodiments of the invention and, together with a general description of the invention given above, and the detailed description given below, serve to explain the principles of the invention.

FIG. 1 is a longitudinal sectional side view depicting one embodiment of an ophthalmic brush apparatus according to the present invention;

FIG. 2 is a longitudinal sectional side view of a hand-held fixation device of the invention.

FIG. 3 is side view of both the ophthalmic brush of FIG. 1 in combination with the fixation device of FIG. 2.

FIG. 4 is a top, plain view of a fixation device in use with a speculum secured to the bony orbit of the eye of a patient.

### DETAILED DESCRIPTION OF THE INVENTION

### Definitions

As used herein, the terms “ultrasound” or “ultrasonic” refer to sound of a frequency higher than, and outside of, the audible range of the human ear. The ultrasound frequency range generally begins at a minimum frequency range of between about 15,000 to about 20,000 cycles per second (cps), or hertz (Hz).

The term “sonic” refers to sound of a frequency that is within the audible range of the human ear, i.e. roughly between 20 Hz to about 20,000 Hz, although the range of frequencies individuals hear can be greatly influenced by environmental factors. Frequencies below 20 Hz are generally felt rather than heard, assuming the amplitude of the vibration is great enough. High frequencies (i.e. between about 15,000 Hz to about 20,000 Hz) are the first to be affected by hearing loss due to age and/or prolonged exposure to very laud noises. That is, a newborn baby can hear up to about 20,000 Hz, but very few adults can. Adult hearing stops somewhere in the range of between about 15,000 Hz to about 20,000 Hz.

Current diagnostic and therapeutic ultrasound devices used in medical procedures typically operate at frequencies between about 1 million Hz to about 20 million Hz (1-20 MHz). In contrast, the ophthalmic apparatus disclosed herein is intended to operate at a relatively low frequency range that encompasses both sonic and ultrasonic frequencies; more specifically, at a frequency range of between about 50 Hz to about 30,000 Hz.

A “circuit board” as defined herein is an insulated board on which interconnected circuits and components such as microchips are mounted or etched. A circuit hoard typically controls the sequence of events needed for proper operation of the apparatus of the invention, including the control and distribution of power to the various electrical components.

“Electrical components” are any elements of the apparatus that run or are powered by electricity. Typically the electrical components of the present invention include, but are not limited to, a circuit board, power source such as a battery, an increase voltage button, a decrease voltage button, an ultrasound generator, an ultrasound transducer, and an on/off button.

The terms “microstreaming” and “acoustic microstreaming” refer to the movement of fluid near and adjacent to microbubbles (e.g., gaseous inclusions within the insonated medium) created by an ultrasonic field. Microstreaming occurs as a result of the action of mechanical pressure changes on the microbubbles. In the context of the present invention, “microstreaming” and “acoustic microstreaming” also can refer to the shear forces associated with microbubbles within eye fluid that are distributed along the surface of the eye. These shear forces, in turn, help remove the superficial corneal epithelium.

As used herein, the term “cavitation” refers to the generation and/or stimulation of bubbles by sound. More specifically, the term “cavitation” is used herein to refer to the interaction between an ultrasonic field in a liquid and in gaseous inclusions (e.g., microbubbles) within the insonated medium. Cavitation of existing microbubbles may be subdivided into two general categories: “stable cavitation” and “inertial cavitation.” “Stable cavitation” is the induction of stable, low-amplitude, resonant oscillations of preexisting microbubbles by low-intensity ultrasonic energy, which, in turn generates local shear forces within the fluid flow (referred to herein, as acoustic microstreaming) near and adjacent to the microbubbles. If the ultrasound intensity is increased, the amplitude of oscillation also rises until the bubble becomes unstable and collapses due to the inertia of the inrushing fluid, giving rise to unstable cavitation or “inertial cavitation.”. The resulting extremes of pressure and temperature within a violently collapsing bubble (one typically more active than those required for the present invention) can be sufficient to initiate free radical generation by hydrolysis of contained water vapor. If bubble collapse occurs in proximity to a fluid/solid interface (for example, the eye surface), shear forces within the medium and high velocity fluid jets can be directed toward the solid structures, i.e., the sub-epithelial corneal surface layers of the eye such as Bowman's layer or the corneal stroma. Extreme inertial cavitation, therefore, is to be avoided when working on a structure as delicate as the human eye, so generally low ultrasonic energy is used with the present invention.

In the context of the present invention, due to the action of bristles tips that can be caused to oscillate together at a low ultrasonic frequency of between about 20 kHz to about 30 kHz, ultrasound-induced stimulation of microbubbles in the eye fluid is intended to cause stable cavitation of these microbubbles, avoiding inertial cavitation. Through the resulting scrubbing action from the shear forces associated with microstreaming, the corneal epithelium can be easily displaced and removed. Alternatively, it is noted mat the power can be adjusted by decreasing the voltage over the piezoelectric unit, such that the amplitude of vibration of the bristles can be reduced, while the frequency is unchanged. This lower energy could be utilized to perform various desired ophthalmologic procedures, when used in conjunction with bristles of various flexural/stiffness characteristics.

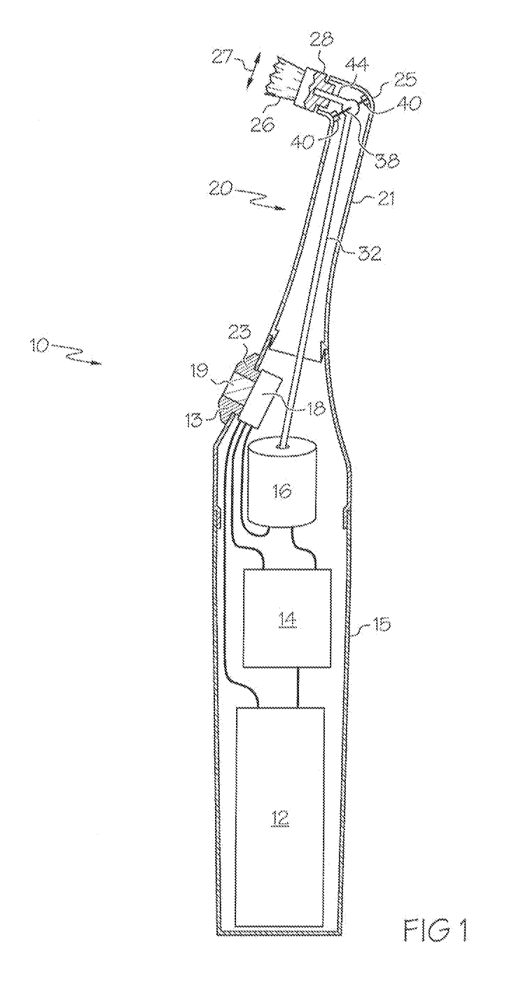

FIG. 1 shows one embodiment of an ophthalmic treatment apparatus 10 according to the present invention. The apparatus 10 typically includes a sonic/ultrasonic motor drive having the capability of either sonically or ultrasonically vibrating a brush during operation. The motor drive can be housed by a handle 15 constructed of a rigid or semi-rigid material, and can include a power source 12 such as an electrical outlet or a rechargeable battery, a sonic/ultrasonic generator 14, a sonic/ultrasonic transducer 16, and a circuit board or transducer control unit 18 to allow manual operation of the apparatus by the user. The circuit board 18 is typically connected to an on/off control button 19 as well as an “up” or increase voltage button 23 and a “down” or decrease voltage button 13 located in the handle for the user to control the device manually, as is known in the electrical arts. The circuit board 18 receives input from the on/off button 19 and the up and down buttons 23, 13 of the apparatus, receives energy when turned “on” from the battery 12, and also connects electrically to the ultrasonic generator 14 and ultrasonic transducer 16. Thus, when the on/off button 19 is placed in the “on” position, an electrical circuit is completed and the battery 12, the generator 14, the up and down buttons 23, 13 and the transducer 16 are operational. Turning the on/off button 19 to the “off” position will break the circuit and these portions of the apparatus 10 will turn off. Alternatively, the Increase and decrease in voltage could be controlled by a foot pedal which connects to the circuit board either wirelessly or through electrical wiring.

Suitable some/ultrasonic generators and frequency matched transducers (e.g. piezoelectric materials), as well as appropriately sized circuit boards and energy sources for fitting into a handle and allowing manual control by the user are well known in the art and will not be described in detail herein. However, the electrical signal produced by the generator 14 should be of a sufficient voltage to drive the transducer 16 via the circuit board/transducer control unit 18, such that the transducer 16 expands and contracts primarily along one axis at or near resonance with the frequency supplied by the generator 14, thereby converting electrical energy into sonic and/or ultrasonic (depending on the amount of voltage supplied) sound waves. The resulting sonic/ultrasonic waves—which are pressure waves—are conducted into, propagated through, and radiated out through an extended resonator arm 32 to a brush head 20 which houses the brush. The brush typically includes a base 28 with bristles 26 secured thereto at the free end of the brush head 20. Preferably the base 28 and bristles 26 of the brush is induced to vibrate approximately in resonance at any frequency selected within the sonic or ultrasonic spectrum.

The extended resonator arm 32 typically attaches at a first, proximal end to the transducer 16, and then extends distally and longitudinally from the handle 15 and into the stem 21 of the brush head 20. The resonator arm 32 is typically an elongated steel member, and is mounted to a steel torsion pin 38 within an angularly curved neck portion 25 of the stem 21 of the ophthalmic brush head 20. In operation, sonic/ultrasonic energy is transferred from the transducer 16 to the resonator arm 32 for achieving an oscillating action about the torsion pin 38. A distal, free end 44 of the resonator arm 32 is attached to the base portion 28 of the brush, which secures the proximal ends of the bristles 26. The combination of the base portion 28 with the attached bristles 26 constitutes the brush, which can be manufactured as a detachable and/or disposable portion of the apparatus. The base 28 typically removably connects to the free end 44 of the resonator arm 32, so that a plurality of base portion attachments with varying diameters and brush properties can be fitted into the end of the brush head 20. The free ends of the bristles 26 are free to vibrate with displacement in a direction indicated by the double beaded arrow 27, generally parallel to the longitudinal axis of the resonator arm 32. The energy supplied by the resonator arm 32 is therefore transferred to the base portion 28 and its corresponding bristle tips 26, which operate within sonic and ultrasonic ranges of amplitude and frequency to produce a bristle tip velocity sufficient to achieve removal of the superficial corneal epithelial tissue and/or a foreign body removal from the cornea or conjunctiva.

As noted above and illustrated in FIG. 1, the resonator arm 32 is fixedly secured to the torsion pin 38. The torsion pin 38 is typically affixed to a collar 40 attached inside the neck portion 25 of the brush head 20. The diameter and length of the torsion pin 38 are selected to provide a spring constant which resonates with the mass and compliance distribution of the remaining free end 44 of the resonator arm 32. The resonant frequency of the torsion pin 38 should be close to the drive frequency of the resonator arm so that the free end 44 will undergo maximum displacement when the transducer 16 is driven at its resonant frequency.

At the free, distal end 44 of the resonator arm 32, i.e. opposite the end that attaches to the transducer 16, are the base 28 and bristles 26 of the ophthalmic brush, head 20. The stem portion 21 of the ophthalmic brush head 20 may be either removably or fixedly attached to the handle 15. The bristles 26 are typically a plurality of contiguous synthetic plastic thin filament bristles densely packed together to provide a soft and pliable, yet substantially continuous abrasive surface at the free end of the base portion 28 of the brush head 20. The bristles 26 can be arranged to form a circular shape, creating a brush head diameter of between about 2 mm to about 10 mm, and more typically between about 6 mm to about 8 mm in diameter. However, the bristles may also form an elliptical shape, or any other suitable shape useful for removing a particular area of the corneal epithelium. The base 28 and bristles 26 do not rotates as in a rotary brush device such as the Amoils brush, but rather oscillate or move in a sinusoidal pattern in the direction of the double arrow 27, as indicated in FIG. 1. The Amoils rotating brush (of the prior art) operates at about 1500 revolutions per minute, and with a typical brush diameter of about 6 mm moves almost linearly in a broad arc, as opposed to the very fine, oscillatory or sinusoidal movements produced by the brush of the present invention.

The bristles 26 can receive the oscillatory vibrations transmitted from the free end 44 of the resonator arm 32 and pass on this energy to the fluid covering the corneal epithelium of the surface of the eye. The individual bristles themselves can be between about 0.15 mm to about 0.40 mm in diameter, extending substantially perpendicularly relative to the axis of the resonator arm 32, and can be made of plastic or other synthetic polymer, microfilaments, or animal hair. The bristles are typically between about 6.0 mm to about 10.0 mm in length, so as to transfer relatively low energy sonic and ultrasonic waves to the eye tissues. Also, individual bristles may vary in length so that the radius of curvature of the surface formed by the free ends of the bundle of filaments is generally formed to define a concave surface to evenly abrade the corneal tissues on the convex surface of the eye.

When low frequency ultrasonic energy is used (i.e. between about 20 kHz to about 30 kHz), oscillating ultrasonic waves are transmitted by the bristles 26 to the corneal eye tissue and act on microbubbles within the eye fluid to induce stable cavitation (as defined herein) of the microbubbles, which can loosen the superficial corneal epithelium. That is, simple volumetric changes in bubbles over time can generate movement within the fluid in proximity to the bubbles, creating mechanical shearing effects that can loosen and remove corneal epithelium. As noted above, in addition to stable cavitation, ultrasonic devices can also produce inertial cavitation, which is the forceful explosion of microbubbles and high liquid velocities. Creation of inertial cavitation is obviously not desired for use on delicate eye tissue. Inertial cavitation extends beyond the tip of the treating device, which in the present invention would be brush bristles. Therefore, excessive ultrasonic energy is neither recommended nor necessary to accomplish the task of corneal epithelial removal, and in fact could result in damage to the Bowman's membrane to which the surface epithelium adheres. The ophthalmic apparatus disclosed herein is intended to operate at a relatively low frequency range that encompasses both sonic and ultrasonic frequencies; more specifically, at a frequency range of between about 50 Hz to about 30,000 Hz.

A mechanically driven sonic brush with bristle stroke of 0.125″ operating at a frequency of 120 Hz will have a peak velocity of about 4 feet per second at the bristle tip, while an ultrasonic brush operating at a frequency of about 20,000 Hz can have a brush bristle stroke of 0.001″ and a peak velocity of about 5 feet per second. However, an ultrasonically-operated brush can have a repetition rate of about 150-200 times greater than the sonic brush, and peak acceleration at the bristle tips can be up to 200 times greater. These factors can cause both stable and inertial cavitation, which can have a much more forceful effect. Thus, to accomplish epithelial removal effectively in different circumstances, it is desirable to use low ultrasonic energy that can be varied to include sonic energy as needed.

For example, corneal epithelial removal in patients who have previously had Laser-in-situ-keratomileusis (LASIK), wherein the LASIK flap can be displaced by a rotary brush (such as the Amoils rotary brush), it would be desirable to use a low frequency, sonically oscillating brush, from about 50 Hz to about 500 Hz. These lower frequencies are efficient at epithelial disruption and removal, yet are unlikely to create microbubbles, cavitation, and/or damage the underlying Bowman's membrane. In routine PRK cases, higher sonic frequencies (from about 500 Hz to about 5000 Hz) may be desirable. Finally, there are situations wherein higher sonic frequencies, i.e. above 5000 Hz and higher, entering into the range of low ultrasonic frequencies be to about 20,000 Hz and up to about 30,000 Hz, would be most efficient, such as in corneal foreign body removal. In these circumstances, a small diameter brush of between about 1.5 mm to about 3.0 mm in diameter can be used to accurately dislodge metal and/or rust from the corneal surface with minimal damage to adjacent tissue.

The amplitude of the bristle vibrations produced may be any in the sonic or ultrasonic spectrum, but are typically between about 0.3 mm to about 1.0 mm. Thee preferred amplitude range is between about 300 microns to about 1500 microns, and preferably about 600 microns. The bristles 26 are induced to vibrate approximately in resonance with the resonator arm 32, such as, but not limited to, a frequency range of between about 50 Hz to about 30,000 Hz (30 kHz). As noted above, the ophthalmic apparatus disclosed herein is intended to operate at a relatively low frequency range that encompasses, both sonic and ultrasonic frequencies (i.e. within a frequency range of between about 50 Hz to about 30,000 Hz.

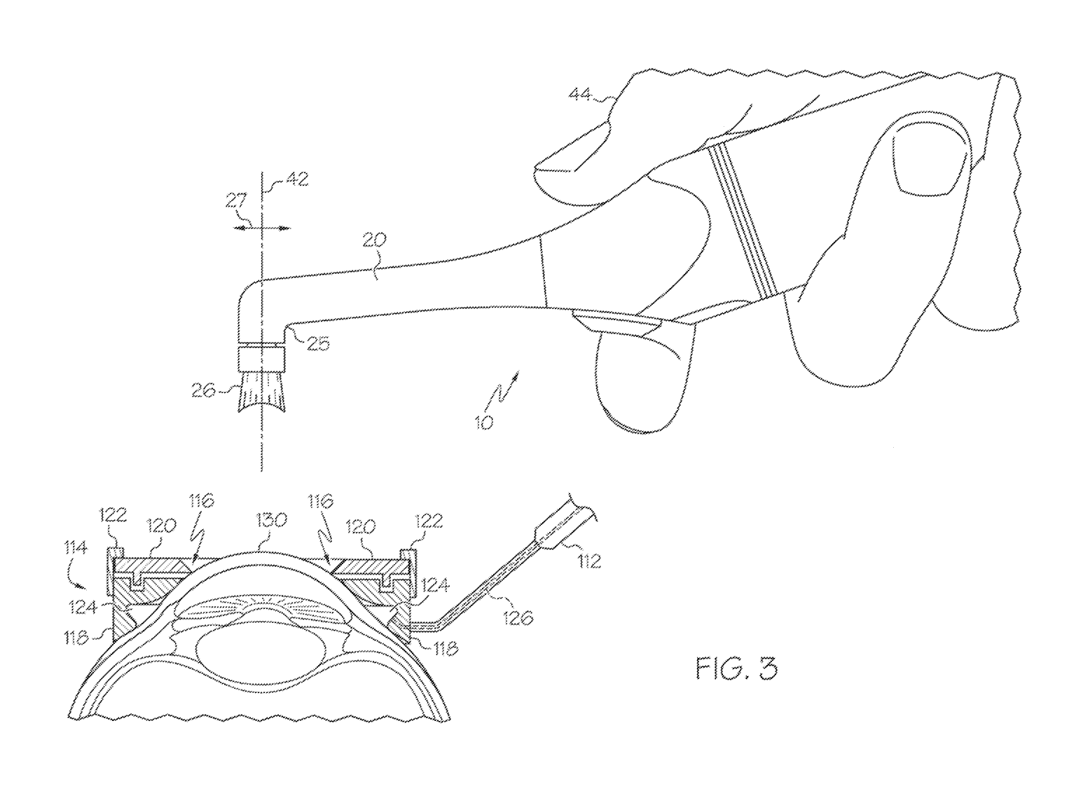

In use, the apparatus 10 is held so that the tips of the bristles 26 are positioned against the surface of the eye, typically over the central cornea. See FIG. 3. As indicated above, the bristles 26 can be arranged on the brush 20 such that the radius of curvature of the surface of the brush bristles defines a concave abrasion surface to evenly abrade the convex corneal epithelial tissues, as illustrated in FIG. 3. The sonic or ultrasonic energy transferred to the bristle tips 26 is intended to assist in removing the superficial epithelial surface of the cornea, with minimal to no damage to the Bowman's layer or underlying stroma. Also, because of the relatively more efficient energy provided by the rapidly oscillating bristles as compared to the movement of rotating ophthalmic brushes, the presence of remaining debris material or ragged edges at the treatment site is significantly lessened, allowing for improved regeneration of the epithelial layer and faster post-operative healing.

In the inventive method for removing corneal epithelial tissue, softening agents such as diluted alcohol or trypsin can be used prior to treatment. The term “trypsin” has been employed generically to include trypsin-like enzymes and acidic trypsins. In passing it may be noted that α-chymotrypsin is not a member of the trypsin family. Trypsins have been isolated in pure form and characterized from widely diverse organisms, including beef, pig, sheep, human, turkeys, shark, crayfish, white shrimp, silkmoth, and even from several strains of streptomyces. In terms of practice of this invention bovine trypsin and porcine trypsin are preferred, both of which are available in crystalline form in sufficiently high purity for pharmaceutical uses. Other trypsins are however contemplated. Preferred compositions of the present invention are then a 15-20% alcohol solution, or aqueous solutions of trypsin in concentrations of 0.01-0.10 mg/ml.

Once the operative surface of the cornea is treated with a diluted trypsin or alcohol solution, the oscillating bristles 26 are typically manually moved over the superficial corneal epithelial layer under relatively light pressure, to further loosen and then remove the corneal epithelium while leaving the underlying corneal stroma layer and Bowman's layer unaffected and intact. Because the layers under the epithelium are fibrous and substantially abrasion resistant, the abrasion is limited to the softer, superficial epithelial cells alone.

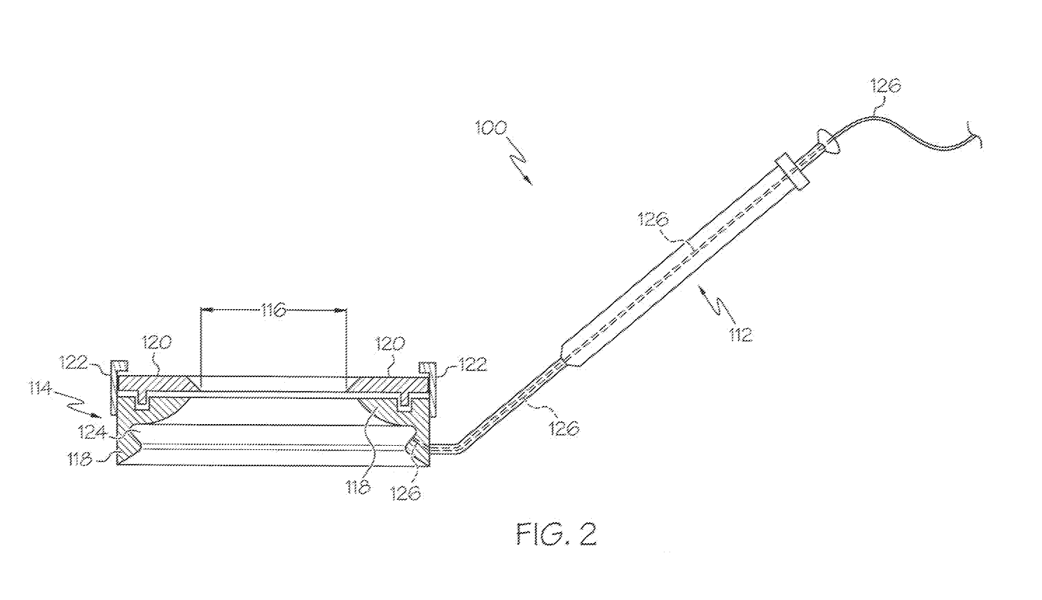

With this type of procedure, it is clearly desirable or necessary to prevent the operative eye from moving, and it would also be beneficial to provide a guide to serve as a demarcation line/area for epithelial removal. Therefore, the present invention can also include the apparatus described above for removing the corneal epithelium in combination with an instrument for fixating the patient's eye during an ophthalmic procedure. See FIGS. 2, 3 and 4. Such a fixation device for use with the present invention can be either vacuum-assisted (to aid in keeping the device on the eye), or vacuum may not be necessary.

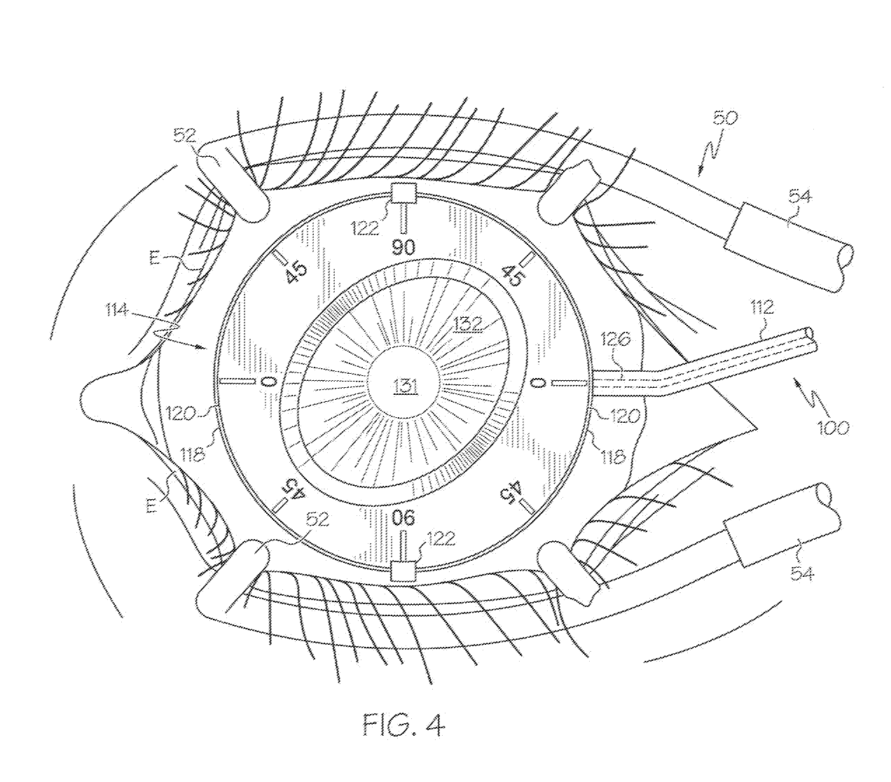

One embodiment of a typical hand-held fixation device 100 for use with the ophthalmic apparatus of the present invention can be seen in FIG. 2, and includes a handle 112 connected to a fixation ring 114. See also FIGS. 3 and 4. The fixation ring 114 can include an upper portion 120 which typically can rotate, and a lower portion 118 for sitting atop the orbit of the eye 130 (as shown in FIG. 3). The lower surface of the bottom portion 118 of the fixation device typically conforms to the sclera-limbus-peripheral cornea curvature of the eye 130. The bottom portion 118 and the rotatable top 120 portion of the fixation ring 114 are typically reversibly connected by a ring clamp 122. Further, the bottom portion 118 can include a circumferential groove or vacuum annulus 124 on the side contacting the eye 130 (see FIG. 3), to which suction or a vacuum 126 can be applied to ensure stable mounting of the fixation ring to the eye 130.

The top, rotatable ring portion 120 can have markings on it for determining the axis of the eye, and an inside edge defining an opening that can be either circular or ellipsoid in shape. See FIG. 4 showing a top, rotatable ring 120 with an ellipsoid opening for treating astigmatism. As best seen in FIG. 3, the inside edge of the top rotatable ring portion 120 opposes the corneal epithelium to define an internal diameter which delineates the boundary of epithelial removal. The inner diameter of the top ring portion 120, in combination with the bottom portion 118, can also define a well 116 when the device 100 is placed on the orbit of the eye 130, so that epithelial-softening/loosening fluid such as alcohol or trypsin (as described above) can be entrapped in order to soften a desired area of the corneal epithelium prior to removal.

As shown in FIG. 3, the ophthalmic apparatus 10 of the invention is positioned and supported by an operator's hand 44 over the eye 130 against and substantially parallel to the cornea. It can be appreciated by viewing FIG. 3 that the surface of the brush head has a concave curvature that is substantially complimentary and corresponding to the convex curvature of the cornea of the eye 130. The operator engages the eye 130 with the brush 26 while the brush is oscillating in the direction of the double arrow 27, which is substantially perpendicular to the axis 42 of the resonator arm of the brush head 20. Tissue removal is relatively rapid, generally occurring in a few seconds. In the past, to ensure access to the underlying stroma, operators have generally removed the epithelium from a significantly larger region of eye than is required. The combination of the ophthalmic apparatus 10 and fixation device 100 of the present invention allows the area of the epithelial tissue removed to substantially match that of the intended treatment site. This allows healing to progress radially inwardly from the epithelium surrounding the treatment area, thereby providing an environment for rapid healing of the epithelium.

The diameter of the opening in the top rotatable ring portion 120 is typically from about 8.0 mm to about 10.0 mm, and preferably from about 8.5 mm to about 9.0 mm for a typical myopia treatment, and (if ellipsoid in shape) typically about 7.0 mm×9.0 mm for an ellipsoid, astigmatic treatment. As noted above, the brush diameter of the ophthalmic apparatus 10 typically is between, about 2 mm to about 10 mm, and more typically between about 6 mm to about 8 mm in diameter. A top rotatable ring 120 having an ellipsoid inner diameter (as seen in FIG. 4) with degree markings can thus easily be placed at the appropriate position to provide a demarcation line or area for removal of epithelium in the correct axis. Alternatively, the fixation device 100 can utilize a non-rotatable top surface, and when used for an ellipsoid astigmatic treatment the device 100 could be positioned onto the globe at the appropriate astigmatic axis.

As illustrated in FIG. 4, a top view of the fixation device 100 shows concurrent use of a speculum 50 having a pair of opposed retractors or blades 52 for holding the eyelids E open against the bony orbit of a patient. The retractors 52 are carried by pair of retractor arms 54 (or equivalent structures), which in turn are secured (not shown) so as to provide adjustability and to securely hold the eyelids E open against the bony orbit. Any suitable biasing mechanism may be used to provide the requisite force for holding the retractors 52 against the bony orbit. As illustrated, the inner diameter of the tops rotatable ring portion 120 of the fixation device 100 is ellipsoid in shape to allow for removal of ovoid shaped during astigmatism correction. The bottom and top portions 118, 120 are reversibly connectable by a ring clamp 122, and the inside edges of the top rotatable ring portion 120 also oppose the corneal epithelium to define an internal diameter which delineates the boundary of epithelial removal. The device can thus easily be placed at the appropriate position to remove epithelium in the correct axis.

The fixation ring 114 can serve as a guide to provide a demarcation line/area for epithelial removal by the sonic/ultrasonic brush of the invention, allowing the brush to provide gentle disruption of the epithelial tissue within the inner diameter of the upper ring 120 of the fixation device. The outer diameter of the fixation ring 114 is typically about 20 mm, and the inner diameter of the top rotatable ring 120, which creates an opening to expose the pupil 131 and the iris 132, has dimensions noted above.

The ultrasonic brush of the invention can yield gentle disruption of the epithelial cells, releasing their attachments to the underlying Bowman's membrane, for easy removal of the corneal epithelium and with less chance for scarring and disruption of the underlying tissue. The brush can also be used for gentle debridement of epithelium for recurrent corneal erosion, and for foreign body removal from the cornea or conjunctiva. Different sized brushes can be provided for such varied endeavors, which can be attached to and removed from the handle portion of the apparatus as needed. The brush bristles can also be varied in length, materials and stillness to maximize the efficiency of the specific procedure being performed.

While the present invention has been illustrated by the description of embodiments thereof in considerable detail, it is not intended to restrict or limit the scope of the appended claims to such detail. Additional advantages and modifications will be readily apparent to those skilled in the art. Accordingly, departures may be made from such details without departing front the scope or spirit of the invention.

What is claimed is:

1. An ophthalmic treatment apparatus for removing the superficial corneal epithelium layer of the eye, the apparatus comprising:
a) a brush for removing the superficial corneal epithelium layer, the brush having a surface adapted to be applied against and substantially parallel to the cornea; and
b) a motor drive for either sonically or ultrasonically vibrating the brush during operation.

2. The ophthalmic treatment apparatus of claim 1, the motor drive comprising:
i) a generator for producing both sonic and ultrasonic energy;
ii) a transducer in electrical communication with the generator; and
iii) a power source for providing power to the generator.

3. The ophthalmic treatment apparatus of claim 2, wherein the motor drive is housed in a handle connected to a stem portion of a brush bead for housing the brush, the handle further including a circuit board to allow manual operation of the apparatus by the user, a resonator arm attached at one end to the transducer and attached at the other end to the brush, the resonator arm further being anchored by a torsion pin secured within the brush bead, wherein either sonic or ultrasonic energy is transferred from the motor drive to the resonator arm and then to the brush for achieving an oscillating action of the brush.

4. The ophthalmic treatment apparatus of claim 1, further comprising an ophthalmic fixating device for stabilizing the eye and for providing a demarcation line/area for epithelial removal by the brush.

5. The ophthalmic treatment apparatus of claim 4, wherein the fixating device comprises a handle connected to a fixation ring, the fixation ring including a bottom portion and a top rotatable ring portion, the bottom and top portions being reversibly connectable by a clamp, wherein the inside edges of the top rotatable ring portion oppose the corneal epithelium and define an internal diameter which delineates the demarcation line/area of epithelial removal.

6. The ophthalmic treatment apparatus of claim 5, the bottom portion of the fixation ring further including a circumferential groove on the side contacting the eye, wherein suction or a vacuum can be applied to the circumferential groove to ensure stable mounting of the fixation ring to the eye.

7. The ophthalmic treatment apparatus of claim 5, wherein when the fixation ring is in contact with the eye the bottom and top portions define a well for placement of a softening solution.

8. The ophthalmic treatment apparatus of claim 5, wherein the inside edges of the top rotatable ring portion form either a circular or an ellipsoid shape.

9. The ophthalmic treatment apparatus of claim 1, wherein the brush comprises a multitude of tightly bundled contiguous thin filament bristles having free ends, the bristles being densely packed together so that the free ends of the bristles make the surface of the brush head substantially continuous.

10. The ophthalmic treatment apparatus of claim 1, wherein the surface of the brush includes a concave curvature substantially complimentary and corresponding to the convex curvature of the cornea.

11. The ophthalmic treatment apparatus of claim 1, wherein the brush is vibrated at a frequency range that encompasses both sonic and ultrasonic frequencies, more specifically at a range of between about 50 Hz to about 30,000 Hz.

12. An ophthalmic treatment apparatus for removing the superficial corneal epithelium layer of the eye, comprising;
a) a brush for removing the superficial corneal epithelium layer and/or for foreign body removal from the cornea or conjunctiva, the brush having a surface adapted to be applied against and substantially parallel to the cornea;
b) a motor drive for sonically or ultrasonically vibrating the brush during operation; and
c) an ophthalmic fixating device for stabilizing the eye and for providing a demarcation line/area for epithelial removal by the brush.

13. The ophthalmic treatment apparatus of claim 12, the motor drive comprising:
i) a generator for producing both sonic and ultrasonic energy;
ii) a transducer in electrical communication with the generator; and
iii) a power source for providing power to the generator.

14. The ophthalmic treatment apparatus of claim 13, wherein the motor drive is housed in a handle connected to a stem portion of a brush head for housing the brush, the handle further including a circuit board to allow manual operation of the apparatus by the user, a resonator arm attached at one end to the transducer and attached at the other end to the brush, the resonator arm further being anchored by a torsion pin secured within the brush head, wherein either sonic or ultrasonic energy is transferred from the motor drive to the resonator arm and then to the brush for achieving an oscillating action of the brush.

15. The ophthalmic treatment apparatus of claim 12, wherein the ophthalmic fixating device comprises a handle connected to a fixation ring, the fixation ring including a bottom portion and a top rotatable ring portion, the bottom and top portions being reversibly connectable by a clamp, wherein the inside edges of the top rotatable ring portion oppose the corneal epithelium and define an internal diameter which delineates the demarcation line/area of epithelial removal.

16. The ophthalmic treatment apparatus of claim 15, the bottom portion of the fixation ring further including a circumferential groove on the side contacting the eye, wherein suction or a vacuum can be applied to the circumferential groove to ensure stable mounting of the fixation ring to the eye.

17. The ophthalmic treatment apparatus of claim 15, wherein when the fixation ring is in contact with the eye the bottom and top portions define a well for placement of a softening solution.

18. The ophthalmic treatment apparatus of claim 15, wherein the inside edges of the top rotatable ring portion form either a circular or an ellipsoid shape.

19. A method for removing the superficial corneal epithelial layer of an eye, comprising the steps of:
a) exposing a corneal surface of the eye;
b) using sonic and ultrasonic energy to vibrate a brush for removing the superficial corneal epithelium layer of the eye, the brush having a surface adapted to be applied against and substantially parallel to the cornea; and
c) applying the surface of the brush to the corneal surface of the eye until the superficial corneal epithelial layer has been removed, while leaving the underlying corneal stroma layer and Bowman's layer intact.

20. The procedure according to claim 19, wherein the step of exposing a corneal surface of the eye includes stabilizing the eye with a fixation device, the fixation device providing a demarcation line/area for epithelial removal by the brush head.
